(19) United States
(12) Patent Application Publication
(10) Pub. No.: US 2011/0130968 A1
(43) Pub. Date: Jun. 2, 2011

(54) Method for computing ligand - host binding free energies

(76) Inventor: Matthew Clark, Wayne, PA (US)

(21) Appl. No.: 12/626,911

(22) Filed: Nov. 29, 2009

(51) Int. Cl. G06F 19/00; G01N 33/48

(52) U.S. Cl. 702/19

(57) ABSTRACT

Methods and systems for computing the binding free energy of a ligand to a host are disclosed. The method entails selecting random poses of a ligand, including translation, rotation, and conformation, and computing the ligand-host interaction energy. The poses are accepted or rejected based on the Metropolis criteria. The entropies of interaction are estimated from the log of the ratio of accepted poses to attempted poses. The free energy of the entire sampling region, and of sub-regions of the binding region, can be accumulated by storing the energies and acceptance ratios for both the entire region and the sub regions. In order to better shape the protein to the ligand, energy minimization of the host atoms in the region of the ligand is carried out in the presence of the lowest free-energy pose of the ligand, optionally preceded by random perturbation of the Cartesian coordinates of the host atoms in the vicinity of the ligand.

### BACKGROUND

The present disclosure generally relates to methods for computational simulation of ligand-host binding, methods for accounting for changes in solvation that accompany ligand binding, methods for modifying the structures of host molecules to improve estimates of binding energies, methods for identifying the preferred macromolecular sites and ligand poses for binding, and methods for evaluating the relative ligand-host binding energies for a series of compounds binding to one or more hosts.

Ligand-host interactions, where the host is commonly a macromolecule such as a protein or ribonucleic acid, are commonly analyzed by “docking” which generally entails sampling a large number of ligand-host interactions. In this method the ligand atomic Cartesian coordinates are defined by the term “pose” which is comprised of the Cartesian translation of the ligand, the conformation produced by rotating chemical bonds, and the rotation of the ligand by Eulerian angles. The docking process samples a large number of poses to locate the pose with the lowest computed ligand-host energy, as computed by molecular mechanics or simpler scoring functions.

The molecular mechanics force fields, and scoring functions commonly used in docking provide a computed interaction energy, which is taken as an approximation of the interaction enthalpy. The docking process does not compute the entropy of the pose, which is required for evaluating the free energy of binding. It is known that the enthalpy of binding does not correlate well to the observed binding.

Observed binding constants, however, are directly related to the ligand-host binding free energy, not the binding enthalpy; K=exp(ΔG/kT), where the observed binding constant K is computed from the exponential of the free energy, ΔG, divided by kT, where T is the measurement temperature, and k is the Boltzmann constant. It has been experimentally verified for ligands of T4-lysozyme, for example, that the binding enthalpy as measured by calorimetry is not related to the observed binding constant. (MORTON et. al.) Therefore accurate computation of the binding of novel compounds requires accurate computation of the binding free energy, which includes both enthalpy and entropy. The binding free energy cannot be computed from the lowest energy pose alone. It is also critically important that the sampling of poses for a free energy computation be truly random. Docking programs do not provide unbiased sampling because they focus on finding the single lowest enthalpy pose by using sophisticated algorithms to quickly focus the sampling to the region where this pose is likely to be found. The method described herein, directly addresses the issue of uniform sampling for calculation of free energy.

One common approximation for computing free energy is the molecular mechanics/Poisson-Boltzmann solvent-accessible surface area (MM/PBSA) method. (WEIS et. al.) This method combines an enthalpy value from molecular mechanics and a computed solvation free energy together with a Poisson-Boltzmann electric potential and a solvent-accessible surface area energy term. While this solvation treatment addresses the potential of displacing solvent and estimates the entropy of solvent displacement and reorganization, it does not directly account for the entropy change of the ligand or host on binding. The method addressed herein applies to the calculation of the entropy of ligand-host association in addition to the terms computed by the MM/PBSA methods.

Such computations can be improved by integrating the energies over an ensemble of poses near the binding pose of interest in order to estimate the entropy of the pose. The mining minima algorithm and related algorithms are examples of the method. The mining minima method, however, uses only local energy minima in its sampling and computation of free energies. In addition the mining minima method relies on the ratio of the total system energies for macromolecular complexes to the uncomplexed components, which involve small differences in large numbers, which introduces significant issues in numerical computations.

Free energy perturbation methods carry out a more complete sampling of states and allow a molecule to gradually grow into, or be removed from, a binding pocket in order to integrate the energy required for the change. In principle, these methods are statistically better than the PB/SA or minima-based methods because the algorithms are more rigorously derived from classical statistical mechanics. However, numeric computational issues are inherent in free energy perturbation methods because these methods are more complex. A significant issue results from the fact that the ligand has a tendency to leave the vicinity of the host as the ligand potential is decreased in the integration cycle. The use of complex constraints and associated methods to remove the thermodynamic effects of the constraints has been developed to attempt to address this problem.

The main limitation of the free energy perturbation methods is that they carry out free energy computations for a pre-selected pose in a pre-selected binding site of the host. While the dynamics-based sampling regimen allows the ligand to move about within the energy barriers defined by the simulation temperature, the ligand cannot cross these barriers to explore different poses. The possible conformations to explore increase dramatically with the number of rotatable bonds in the molecule. Accordingly, fully sampling the conformations of a flexible molecule is very computationally intensive. In contrast, the method described herein allows sampling of multiple conformations of the ligand, sampling of multiple possible binding poses, and sampling of multiple structural states of the host at comparable or improved speeds compared to the free energy perturbation methods.

The method described herein shares some common aspects with the grand canonical sampling approach of Guarnieri in which small organic fragments are sampled about the host, which is defined to be a macromolecule. In both methods, the criteria for accepting or rejecting a movement of the ligand is dependent on the energy of the new pose. In the grand canonical, however, ligands may be inserted or deleted from the system and the probability of accepting a change is also dependent on the total number of fragments in the system. In the approach described herein the sampling is not grand canonical since there is only a single ligand in the system at any time and there is no dependence on the number of particles in the system. In addition, the method described herein includes computation of the enthalpy and entropy of conformational sampling, while the method described by Guarnieri teaches only the computation for rigid fragments.

The method described herein shares some common aspects with the microcanonical ensemble simulation in that a single ligand is repeatedly sampled about the host, and statistical thermodynamics values are computed. However in the microcanonical method all states have equal energies, while in the method described herein the states have energies defined by the ligand-macromolecular interaction.

The method described herein is different than the normal canonical ensemble that has been widely used for molecular simulations in that there is only one instance of the ligand that is sampled about the host. In typical canonical simulations a pre-defined population of ligands is sampled about a macromolecular surface and the simulation creates a configuration of the ligand geometries about the surface. The canonical simulation cannot easily provide a free energy of binding of the ligand to the protein, so that the method described herein is a significant advance over the canonical simulation.

In sampling fragments or flexible molecules about a host surface, previous methods commonly have used a rigid host for the sampling. Using a rigid host greatly simplifies computational complexity but creates artifacts in the free energies since the host structure is known to change to accommodate various ligands. For example, using a rigid host model results in the binding site sometimes being too small to accommodate a tight-fitting ligand that could fit if the host were to move a small amount. The method described herein includes a process for adjusting the host structure to accommodate the ligand.

For free energy computations, the role of a solvent may be considered. For example, in a biological setting for drug design, the role of a solvent is particularly relevant. The entropy and enthalpy for creating a cavity in the solvent, filling it with a ligand, and re-ordering the solvent about the ligand has been explored experimentally and theoretically.

Typically, simulations use either a continuum solvent approach or an explicit water model. Continuum solvent approaches compute the effect of a generic bath of water. This can improve results, but does not provide insight into specific water interactions in the binding site.

Explicit water models, whether modeled as populated periodic boxes or droplet models, bathe the host in bulk water. The bulk water does not permit the ligand to freely move between different poses and conformations. As such, sampling of ligand binding is inhibited. In addition, the total possible number of individual microstates in an explicit liquid state simulation can be quite large. The method described herein uses a explicit simulation of the solvent to correct the free energies of the simulated ligand-host interaction while at the same time allowing full sampling of ligand poses and conformations.

### SUMMARY

It is to be understood that this disclosure is not limited to the particular systems, devices, and methods described, since these may vary. It is also to be understood that the terminology used in the description is for the purpose of describing the particular versions or embodiments only and is not intended to limit the scope.

Unless defined otherwise, all technical and scientific terms used herein have the same meanings as commonly understood by one of ordinary skill in the art. Although any methods, materials, and devices similar or equivalent to those described herein can be used in the practice or testing of embodiments, the preferred methods, materials, and devices are now described. All publications mentioned herein are incorporated by reference. Nothing herein is to be construed as an admission that the embodiments described herein are not entitled to antedate such disclosure by virtue of prior invention. As used herein, the term “comprising” means “including, but not limited to.”

In an embodiment, a computer-implemented method of analyzing a host and potential ligand molecule for binding interactions may comprise selecting a binding region for sampling, which may be determined from known co-crystallized ligands of the host, selecting a random conformation of the potential ligand, selecting a random Cartesian position in the binding region, and selecting a random angular orientation of the potential ligand. A selected combination of conformation, Cartesian position, and rotation of the ligand is referred to as a “pose”. The interaction energy of the selected pose with the host is then evaluated using molecular mechanics force fields known to those with ordinary skill in the art of molecular computations.

A given ligand pose may have associated with it intramolecular attractions or repulsions, which collectively are termed the “conformational energy”. These energies can be computed by methods known to those with ordinary skill in the art of molecular computations.

The energy required to displace the solvent from the ligand and the host is computed by performing the simulation process described herein with the selected solvent molecule. The solvent may be water, or any other molecule. The solvent free energy is computed for the selected binding region, and also for sub-regions in the binding region

When simulating solvent binding, the interaction of solvent molecules may be especially strong in some regions of the host. Solvent molecules may be included as part of the structure of the host in these regions. Such molecules are referred to as “structural” solvent molecules.

The binding free energy is computed for the entire sampling region, and also for discrete sub-regions of the sampling region by tracking the enthalpy and entropy separately for each discrete sub-region desired. In one embodiment this is carried out by using a grid to separate the binding region into cells, although other divisions could be used.

When binding of a ligand pose is simulated, first the host-ligand interaction energy is computed using a molecular mechanics force field, and added to the conformational energy for the pose. Then a correction for solvation is estimated by subtracting the solvation energies corresponding to the individual cell that each atom of the potential ligand occupies. This process results in a solvation and conformational energy corrected interaction energy. The pose is accepted or rejected using the normal Metropolis Monte Carlo criteria based on this corrected energy.

In order to create a more accurate fit of the ligand to the host, the host structure is periodically adjusted based on the forces created by the lowest free energy pose. The atoms of the host as well as any structural solvent molecules are moved in response to the ligand in order to optimize the host structure to best fit each ligand.

The procedure of selecting a pose, selecting a host structure, evaluating the energy terms, and deciding whether to accept the pose is carried out repeatedly to amass a statistical thermodynamic evaluation of the binding interaction between the host and the potential ligand. The binding free energy is computed by using the corrected, Boltzman averaged interaction energy as the enthalpy, and the logarithm of the ratio of accepted poses to attempted poses as the entropy. The binding free energy is then computed as the difference between the enthalpy and entropy.

In an embodiment, a system for analyzing the host and potential ligand binding may include a processor, and a processor-readable storage medium in communication with the processor. The processor-readable storage medium may include one or more programming instructions for performing a method of analyzing the host-ligand binding. The method may include selecting a potential ligand, randomly computing a pose, and the associated binding interaction using a molecular mechanics force field. The method may include repeating the random selection of a pose, and computation of energy. The method may also include evaluation of the binding free energy at various points in the binding site by dividing the binding site into smaller regions using a Cartesian grid, and performing the statistical thermodynamics statistics on each grid cell. The process then will involve writing or printing out the statistical thermodynamic quantities for the entire binding site, and for each cell in the grid.

### DESCRIPTION OF THE DRAWINGS

Aspects, features, benefits and advantages of the present invention will be apparent with regard to the following description and accompanying drawings, of which:

FIG. 1. Shows an example grid, 1, with host atoms, 5, and their location, 2, on the grid. The unavailable cells, 4, because they contain a host atom, are hashed, while the available cells, 3, are shown as open. In the sampling process, an available cell is selected, and a random position inside the cell is computed. The selection of a random point within the chosen cell makes the simulation largely independent of artifacts due to the choice of grid size and position. A counter for the number of attempts is incremented for both the entire sampling region, and the particular cell that contains the insertion point.

FIG. 2 shows a diagram of one type of sampling region defined by known ligands for the protein Factor Xa.

FIG. 3 Shows another type of sampling region defined by a square box around the binding site of Factor Xa. The regions marked as unavailable are apparent by the gaps in the available region, denoted in blue, where the marked sampling region is close to the host.

FIG. 4. Shows the steps involved in the simulation. A free sampling cell is chosen, and then a random position in the cell is computed. Next a random rotation is made to the ligand; then a conformation is chosen by randomly changing the rotations of the single bonds. If the random conformation is not acceptable because atoms overlap, new random conformations are chosen until the resulting molecule does not have overlapping atoms. Next the ligand-host interaction energy is computed using a molecular mechanics force field, In one implementation this is the AMBER force field, which is well known to those with ordinary skill in the art. This energy is corrected by summing the solvation correction associated with each atom of the ligand. The energy is computed using equation 1.

FIG. 5 shows the equilibration of the enthalpy and free energy plotted versus the number of simulation steps for the p38 MAP kinase allosteric binding site. The equilibration is rapid and efficient.

FIG. 6 is a block diagram of an exemplary system that may be used to contain or implement the program instructions according to an embodiment.

### DETAILED DESCRIPTION

The following terms shall have, for the purposes of this application, the respective meanings set forth below.

A “host” refers to a molecule or collection of molecules. Thus, although the term typically refers to proteins, ribonucleic acids, structures formed of both nucleic acid and protein, carbohydrates, structures formed of two or more of the aforementioned, and the like, it can also refer to structures formed with any other molecule or molecules including lipids. Hosts are used in the method described herein with reference to 3D structure comprised of a set of Cartesian coordinates for each atom. Such structures are typically generated by X-ray diffraction studies, which have generated 3D structures for thousands of hosts. However, structures can be produced by other methods such as computational methods or computational methods supplemented by other data such as NMR data.

A “ligand” refers to a molecule or collection of molecules interacting with a host molecule. Generally the ligand has fewer atoms than the host and is the entity that is rotated, translated, and conformationally sampled in the process of computing the binding free-energy for the host-ligand complex.

A “sampling site” is a molecular binding site for a host at which a molecular fragment of a particular molecular species can, as a practical matter, reside. Sampling sites are determined by defining a volume that contains a relevant part of the host in Cartesian space.

A “pose” is a geometrical transformation of the atomic coordinates of a ligand molecule created by the combined operations of translation in Cartesian space, rotation of the molecule by Eulerian angles, and molecular conformation created by rotating chemical bonds. It is implicit that the selected rotation of chemical bonds does not result in a configuration where any non-bonded atoms of the molecule are closer than their van der Waals radii.

A “structural” solvent molecule is a solvent molecule treated as part of the structure of the host. The starting pose of a structural solvent molecule may be assigned, among other means, by reference to experimental data such as a crystallographic structure or computationally.

“Conformational energy” is the energy derived from intramolecular interactions of the atoms of a molecule. This is computed using molecular mechanics force field methods, such as AMBER (CORNELL et. al.) or other common force fields well known to those with ordinary skill in the art.

In an embodiment, simulation may be performed utilizing a Monte Carlo algorithm. A Monte Carlo algorithm is a numerical computational algorithm that simulates the behavior of a physical system, such as a molecular model. Monte Carlo algorithms are stochastic in that they depend upon the use of random (or pseudo-random) numbers. In particular, the Monte Carlo algorithm used in the simulations described herein may determine the ligand and solvent poses.

In an embodiment, the simulation may use the Metropolis sampling criteria, whereby a randomly selected pose is either “accepted” or “rejected” for inclusion in the statistical analysis based on the exponent of a computed interaction energy.

To make the simulation more efficient by avoiding attempted poses inside the protein structure, a grid is used to define the binding region and define sub-regions or cells that are filled by the protein and cells that are available for sampling. This grid can be defined as either the union of the volumes of a set of template ligand atoms, or as a simple paralleliped defined by a center, and the extent in the x, y, and z dimensions. Each grid cell in the sampling region is compared to the protein structure, and if the volume of any atom of the protein structure falls within the grid cell, the cell is marked as unavailable for sampling. The translation aspect of the pose is then required to fall within an available cell in the sampling region.

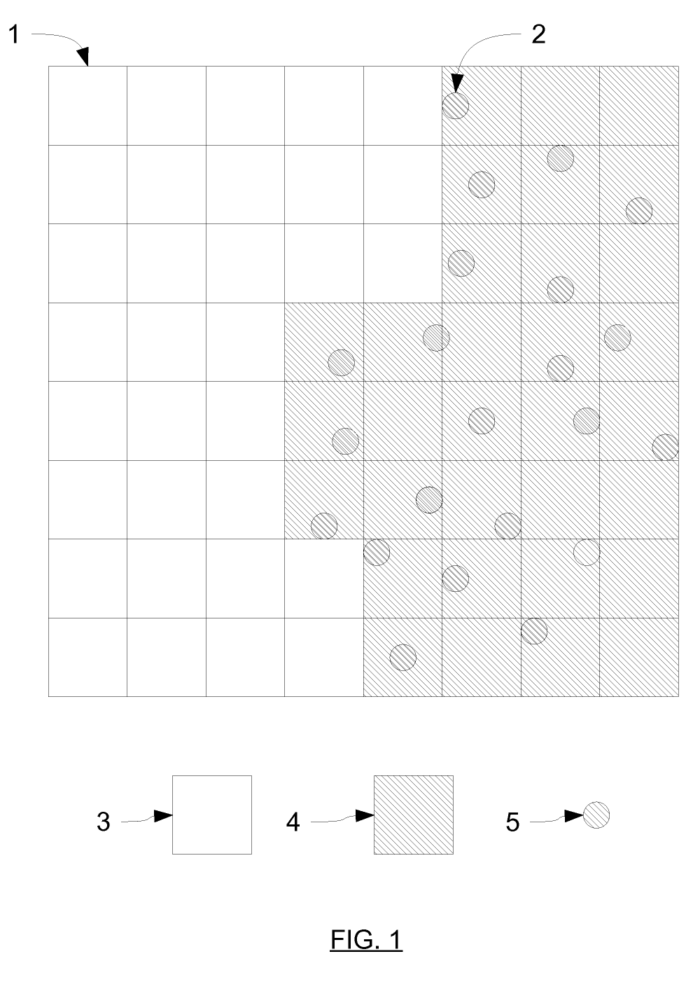

FIG. 1 Shows an example grid. 1 is the grid, and 2 shows a protein atom in the grid. The unavailable cells, because they contain a protein atom are hashed as shown in 4 while the available cells are open as shown in 3. The protein atoms are 5. In the sampling process, a free cell 3 is selected, and a random position inside the cell is computed. The selection of a random point within the chosen cell makes the simulation largely independent of artifacts due to the choice of grid size and position. A counter for the number of attempts is incremented for both the entire sampling region, and the particular cell that contains the insertion point.

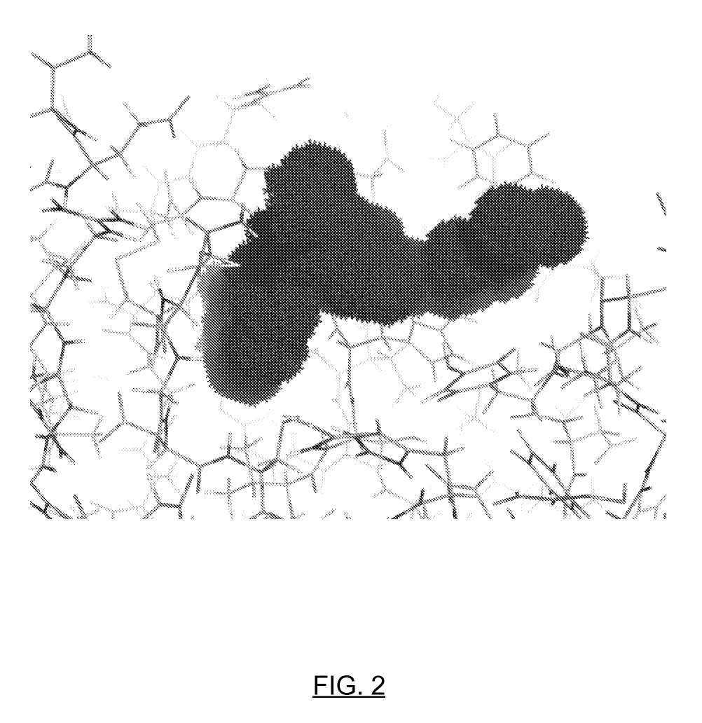

FIG. 2 shows a diagram of one type of sampling region defined by a known ligand for the protein Factor Xa. The geometric center of potential ligands is sampled within this region.

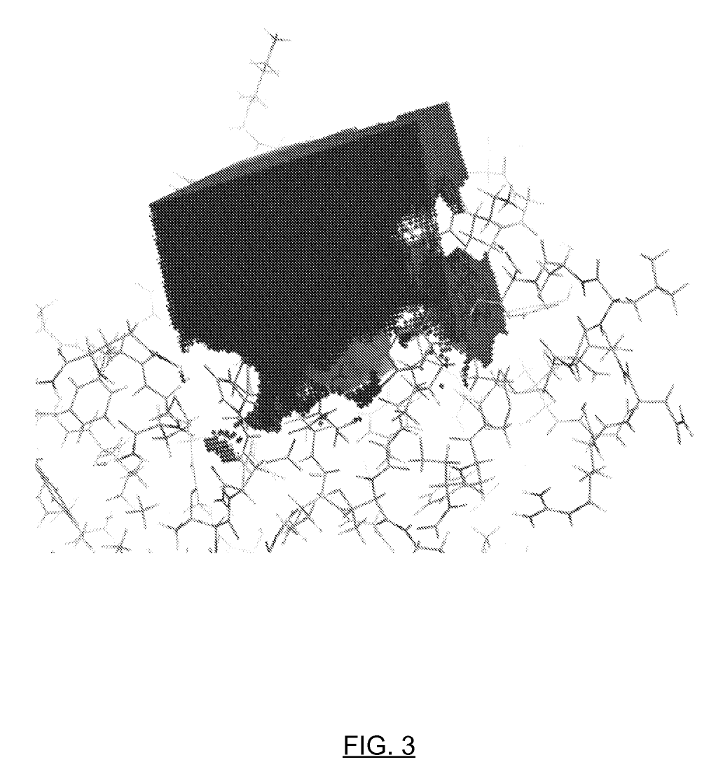

FIG. 3. Shows another type of sampling region defined by a square box around the binding site of Factor Xa. The regions marked as unavailable are apparent by the gaps in the available region, denoted in blue, where the marked sampling region is close to the protein.

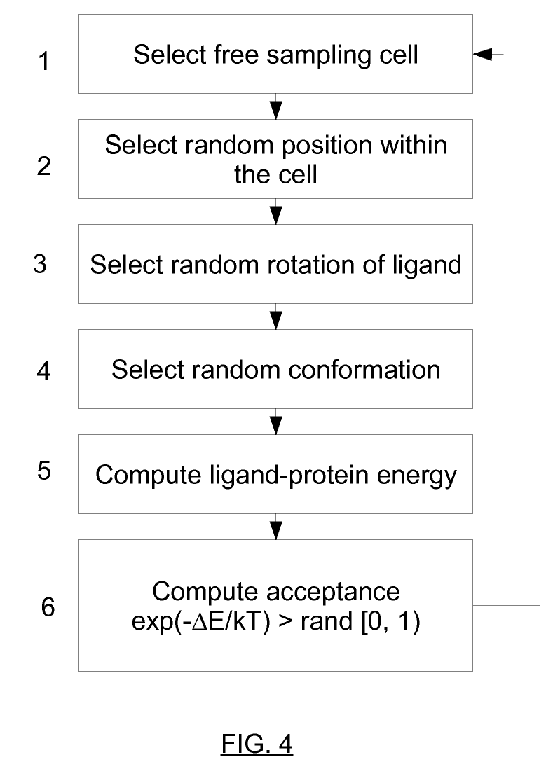

FIG. 4. Shows the steps involved in the simulation. A free sampling cell is chosen 1, and then a random position in the cell is computed. Next a random rotation is made to the ligand; then a conformation is chosen by randomly changing the rotations of the single bonds by incrementing by a random angle between 0 and 360 degrees. The altering of rotation of some bonds, such as amide bonds, may be omitted. The random altering of rotation of double bonds may be restricted to angles near 180 degrees. Rings may be sampled by selecting a ring-closure bond and requiring that the rotation of bonds in the ring maintain the distance of the atoms in the ring-closure bond within a selected tolerance, for example within 10% of the original distance.

The ligand-host interaction energy and the conformational energy of a particular pose of the ligand are computed using a molecular mechanics force field, in one implementation the AMBER force field (CORNELL et. al.) which is well known to those with ordinary skill in the art. Other force fields or scoring functions would suffice as well. The energy is computed using equation 1.

$$E = \sum \frac{A}{{r}_{ij}^{12}} - \frac{B}{{r}_{ij}^{6}} + \sum \frac{k {q}_{i} {q}_{j}}{{r}_{ij}} + {E}_{conformation} \qquad ( 1 )$$

In equation 1 the sum is performed over all pairs of protein atoms i and ligand atoms j, using the distance between them rᵢⱼ, and their electrostatic charges qᵢ and qⱼ and the columbic energy constant k. Distance cutoffs may be used to eliminate atom pairs farther than a given distance.

The conformational energy, E_{confirmation}, is the conformational energy of the guest computed using the AMBER force field, relative to either the lowest energy conformation, or as another option, the energy of the starting conformation. This term serves to weight the interaction energy by the conformational strain of the guest so as to lessen the overall binding energy if the ligand has adopted a conformation with a high energy.

The total energy of equation 1 is used to accept or reject the pose using the standard Metropolis Monte Carlo process. In this process a random number between 0 and 1 is selected. The probability of accepting the pose is computed using the Metropolis criteria shown in equation 2 and compared to a random (or pseudo-random) number generated in the range [0, 1). If the Metropolis criterion is greater than this random number the pose is accepted. If the embodiment of this method involves a biased selection of a particular part of the sampling region for the sampling Cartesian coordinates, the probability computed in equation 2 can be adjusted proportionally to the amount of bias, as is normally done in a Metropolis Monte Carlo simulation. In equations 2, 3, and 4 k is the Boltzmann constant; T is the simulation temperature, typically 300K.

probability=max(exp(−E/kT),1) (2)

If the insertion is accepted a counter for the total number of accepted poses is incremented for both the entire sampling region, and for the particular sub-region or cell that contained the insertion point.

For the simulation the process outlined in FIG. 1. is carried out repeatedly. The entropy contribution of limiting the rotation and conformational freedom of the pose in the sampling region is computed using the formula of equation 3.

$${Entropy}_{rotation - conformation} = kT * exp ( \frac{acceptinsertions}{attemptedinsertions} ) \qquad ( 3 )$$

The translational entropy is computed by comparing the volume of the sampling region to the average volume per molecule of a 1M solution, 1660 Å³. The translational entropy is computed using equation 4.

Entropyₜᵣₐₙₛₗₐₜᵢₒₙ=kT*exp(sampling volume/1660) (4)

The entropy contribution of equation 4 is computed both for the entire sampling region, and for each individual sampling cell. For example if the sampling region available volume is 214 Å³. The translation entropy of limiting the translation volume from 1660 to 214 Å³ is 1.2 kcal/mol. For a sampling cell 0.25 Å per side the translational entropy is 6.9 kcal/mol. The free energy integrated over the binding site is thus given by equation 5.

$$Δ {G}_{binding} = \frac{\sum E * exp ( - E / kT )}{\sum exp ( - E / kT )} - {Entropy}_{rotation - conformation} - {Entropy}_{translation} \qquad ( 5 )$$

In equation 5 the first term is the Boltzmann-weighted enthalpy summed over all of the accepted poses, and the second and third terms are the entropy as computed in equations 3 and 4. The same equations can be applied to each cell in the simulation region, which typically is a cube 0.25 A per side. The entropy of each cell is computed from the ratio of accepted to attempted insertions as shown above, and the enthalpy component of the binding free energy is computed by either the lowest energy observed for each cell or by computing the Boltzmann-weighted energy for the poses accepted in the cell as shown in the first term of equation 5.

In order to correct for the energy of displacing solvent from the host binding site, the simulation process is carried out using a solvent molecule, which is typically water but could be any molecule. The free energies, or alternatively the enthalpies, are stored for each grid cell. These energies are then saved to a computer file and used to correct the free energy of ligand binding as described below.

This correction is carried out for each sampled pose by first determining which cell each ligand atom falls in, and retrieving the stored energy of binding of the solvent in that cell from the saved grid. The solvation penalty for each atom is then computed using equation 6.

$${E}_{solvation , atomi} = min ( {E}_{celli} + {E}_{offset} ) \\ {E}_{solvation} = {\sum }_{atoms} ( {E}_{solvation , atomi} ) \qquad ( 6 )$$

Where E_{cell i} is the computed energy of the solvent in cell i. E_{offset} is an offset for the enthalpy of the pure solvent, for example the enthalpy of pure water is 10 kcal/mol. However other values may be used to adjust the simulation to better agree with experiment.

In one implementation, the energy E_{cell i} used in Equation 6 is the enthalpy, and the sum of the solvation enthalpies over all atoms is computed. This correction is then added to the overall enthalpy of the pose that is computed in Equation 1 and subsequently used in the Metropolis criteria of Equation 2. In an alternative implementation, the energy E_{cell i} used in Equation 6 is the free energy, and the sum of solvation free energies over all atoms is computed. This correction is then added to the overall free energy of the molecule as computed in Equation 5.

In order to optimize the protein to create the best fitting structure, the lowest free energy pose is periodically evaluated. In one embodiment the interval is every minute of simulation time, however the interval could also be based on the number of simulation steps. The forces on the protein and ligand atoms are then computed, using the first derivative of equation 1 with respect to the distance rᵢⱼ. These forces are then summed for all ligand-protein atom pairs i,j to compute the total force on each atom. The protein atoms are then moved based on these forces. In order to avoid excessive adjustments the atoms are only moved if the force is greater than some threshold value, typically from 0 to 1 kcal/mol-Å. The change in each atom position is computed by multiplying the computed force vector for the atom by a pre-selected step size, typically in the range from 0.01 to 0.05. If the change in position is greater than a pre-selected maximum movement, typically 0.1 to 0.25 Å, the change in position is scaled to be the maximum movement. Then this movement vector is added to the atom position to move it in the direction of the force imposed by the ligand.

In addition to the minimization, the Cartesian coordinates of the protein atoms in the vicinity of the ligand may randomly perturbed by an amount similar to a bond length, typically 0 to 0.5 Å, followed by minimization as described above. A Gaussian distribution centered at zero may also be used for the amount of perturbation. This process allows the protein atoms to move to different energy minima, in a process similar to that called “random incremental pulse search” (FERGUSON et. al.) used in conformational search of small molecules.

In addition to the host-ligand forces, the intra-molecular forces of the protein atom may be computed so as to include the energy of moving the protein to better fit the ligand during the adjustment of atom positions. These forces may include the non-bonded terms described by equation 1, as well as the typical bond length, bond angle, and torsion force field terms well known to those with ordinary skill in the practice of molecular structure computations. Using the intra-molecular forces prevents the macromolecular structure from becoming unrealistically distorted when the atoms are moved in response to the forces from the ligand.

In an alternative implementation, the motion of each protein atom may be limited to a pre-selected distance relative to the position in the starting structure in order to prevent unrealistic changes to the protein model from the ligand forces. This limitation is typically around 0.5 to 1 Å movement.

In those cases in which the macromolecular structure contains structural solvent molecules, movement of these solvent molecules may be included in the optimization of the protein structure.

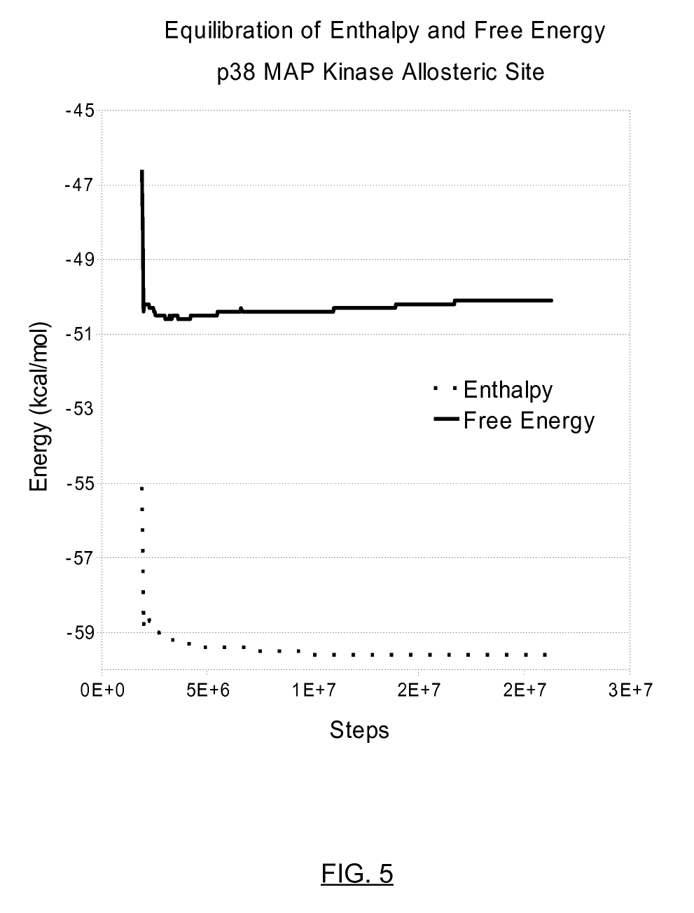

The equilibration properties of the method described herein are shown in FIG. 5. The enthalpy, and free energy computed using equation 5 for the entire sampling region is plotted versus the number of simulation steps. The values converge quickly after 5 million steps, however the increasing quality of the entropy estimate due the larger statistical sample of the ratio of accepted to attempted steps causes the free energy to slowly rise 0.5 kcal/mol. In general the equilibration is efficient enough for results within a few cpu-hours of computation.

One advantage of the method described herein is that the simulation is free from assumptions about the starting pose or nature of binding site. Free energy perturbation methods may compute the binding free energy of a pre-selected pose. In contrast, the simulation described here samples and computes the free energy of molecular fragments without any assumptions other than proximity to the host. This makes the method well adapted to locating novel binding sites and poses. In addition, the accuracy of the free energy computation is increased because the sampling includes large diversity of poses as compared to perturbation calculations on a limited number of poses near a chosen starting pose.

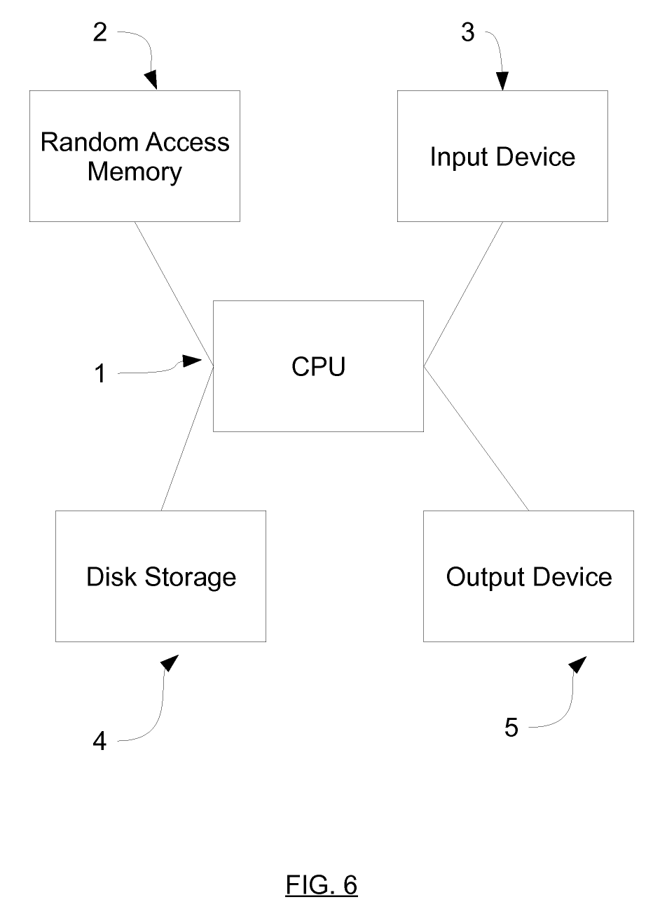

FIG. 6 is a block diagram of an exemplary system that may be used to contain or implement the program instructions according to an embodiment. Referring to FIG. 7, a CPU, 1, is the central processing unit of the system, performing calculations and logic operations required to execute a program. Random access memory (RAM), 2, constitutes an exemplary memory devices or storage media.

Disk drives, 4, serve to store program files and the results of the computations. These disk drives may include, for example, external or internal DVD drives, CD ROM drives, or hard drives. As indicated previously, these various disk drives and disk controllers are optional devices.

Program instructions may be stored in the RAM, 2. Optionally, program instructions may be stored on a computer readable storage medium, such as a compact disk, a digital disk, a memory or any other tangible recording medium.

An optional output device, 5, may permit information to be presented in audio, graphic or alphanumeric format. The output device may also produce a tangible hard-copy of the results of the computation.

In addition to the standard computer-type components, the hardware may also include input devices, 3, which allow for receipt of data from other computers or human users.

An embedded system may optionally be used to perform one, some or all of the operations described herein. Likewise, a multiprocessor system may optionally be used to perform one, some or all of the operations described herein.

It will be appreciated that various of the above-disclosed and other features and functions, or alternatives thereof may be desirably combined into many other different systems or applications. It will also be appreciated that various presently unforeseen or unanticipated alternatives, modifications, variations or improvements therein may be subsequently made by those skilled in the art which are also intended to be encompassed by the disclosed embodiments.

1. A method for computing the binding free energy of a ligand molecule to a host molecule by a process of randomly selecting a translation coordinate in a selected region near the host, rotation angles, and conformation of the ligand,
computing the interaction energy between the protein and ligand,
deciding whether to accept or reject the ligand pose using a Metropolis criteria,
counting the number of poses generated, and the number of poses accepted,
computing the binding entropy from the ratio of accepted to attempted poses,
and the binding free energy by subtracting the entropy from the computed enthalpy.

2. The method of claim 1, wherein the host structure is periodically adjusted based on the forces induced by the ligand so that the host atoms are moved to better accommodate the ligand by moving in the direction of the computed force on each host atom.

3. The method of claim 1, wherein the entropy, enthalpy, and free energy are computed for sub-regions of the sampled binding site.

4. The method of claim 1 wherein the computed free energy is corrected by subtracting the energy of a solvent in the location of each ligand atom using a simulation of the solvent to pre-compute the energies in the binding site.

5. The method of claim 1 wherein the computed energy is corrected for the conformational energy of ligand poses.

6. The method of claim 1 where the sampling region is created by either the union of the volumes of known or proposed ligands, or by defining an explicit sampling region.

7. The method of claim 1 where the results of the computation are printed out to paper output, or displayed on graphical display screens.
